(19) United States
(12) Patent Application Publication
(10) Pub. No.: US 2025/0207079 A1
(43) Pub. Date: Jun. 26, 2025

(54) SYSTEMS AND METHODS FOR CYANOBACTERIA INDOOR AIR BIOFILTRATION TO REDUCE AIR HANDLING ENERGY CONSUMPTION

(71) Applicant: VerdeTerra LLC, Atlanta, GA (US)

(72) Inventor: Mark SNYDER, Atlanta, GA (US)

(21) Appl. No.: 18/984,125

(22) Filed: Dec. 17, 2024

Related U.S. Application Data
(60) Provisional application No. 63/614,038, filed on Dec. 22, 2023.

(51) Int. Cl. C12M 1/34; C12M 1/00; C12M 1/04; C12M 1/36; F24F 8/175

(52) U.S. Cl. CPC C12M 41/34; C12M 23/34; C12M 27/04; C12M 29/22; C12M 41/06; C12M 41/12; C12M 41/26; C12M 41/48; F24F 8/175

(57) ABSTRACT

Systems and methods for cyanobacteria indoor air biofiltration to reduce air handling energy consumption are disclosed. A system may include: a heating, ventilation, and air conditioning (HVAC) system comprising an air valve that controls a flow of conditioned, untreated air from an enclosed area; and a biofiltration device comprising: a controller that interfaces with the air valve and controls the air valve to open or close; an air inlet that receives the untreated air from the air valve when the air valve is in an open position; a bioreactor comprising a bio-organism; an air bubble mixing system that receives the untreated air and diffuses the untreated air into the bioreactor, wherein the bio-organism is configured to consume CO₂ via photosynthesis and to release treated air; and an air outlet that receives the treated air from the bioreactor and expels the treated air to enclosed area.

### BACKGROUND OF THE INVENTION

### 1. Field of the Invention

Embodiments are generally directed to systems and methods for cyanobacteria indoor air biofiltration to reduce air handling energy consumption.

### 2. Description of the Related Art

People spend 80-95% of their lives indoors, where air quality is 4-5 times worse than outdoors. Indoor CO₂ levels have been shown to reach unhealthy levels in occupied meeting rooms and classrooms, even with existing mitigation technologies. The effects of increased CO₂ include reduced cognitive performance and respiratory illnesses, such as asthma. This is exacerbated by the climate crisis as Heating Ventilation and Air Conditioning (HVAC) systems are becoming essential, and buildings are better sealed. HVAC systems account for 38% of a building's energy consumption and 12% of global consumption.

To mitigate the indoor air quality problems, current HVAC systems are designed to introduce a percentage of untreated outdoor air via an energy-inefficient process of dehumidification and temperature control. The climate crisis exacerbates the problem by creating a negative feedback cycle of the increased need for HVAC systems, driving increased energy expenditure, climate impacts from energy generation, and ultimately more harmful air quality.

Other solutions involve the use of filters that include activated charcoal or zeolite. Charcoal is cheap but limited in capacity and requires frequent replacement. Zeolite traps CO₂ in the International Space Station but requires the vacuum of space to then clean the filter. Active systems such as ionization, ultraviolet, and photocatalytic oxidation can remove some volatile organic compounds but increase CO₂ levels and may generate ozone in the process.

### SUMMARY OF THE INVENTION

Systems and methods for cyanobacteria indoor air biofiltration to reduce air handling energy consumption are disclosed.

Embodiments may utilize photobioreactors as air scrubbers with cyanobacteria to capture CO₂ and other Volatile Organic Compounds (VOCs) such as, but not limited to: CO₂, CO, SOx, NOx, PM2.5, and PM 10 for air purification indoors. Embodiments may use the CO₂ scrubber Spirulina platensis, which is 10-20 times more effective at removing CO₂ than any terrestrial plant, or other microalgae species such as, but not limited to: Chlorella vulgaris, scenedesmus, or nannochloropsis. Embodiments may provide full automation and may only require a biomass collection service to keep cost and maintenance intervals in line with existing HVAC services.

Embodiments may comprise a control system that directly interfaces photobioreactors with HVAC systems to reduce energy consumption by subverting the demand on the outside air injection requirements. For example, by providing treated air having an air quality that is an improvement over the dirty or untreated air, embodiments may reduce the demand for outdoor air being pumped in to refresh interior air quality. This outdoor air requires temperature and humidity conditioning, all of which are energy intensive processes. Thus, by treating already conditioned air, embodiments reduce this burden caused by introducing outdoor, untreated air, and thus reduce energy usage by the HVAC system.

Embodiments may be applied to multiple industries and scales to suit a variety of uses. The interior air scrubbing of the system may improve the indoor air quality by removing CO₂ and other VOCs from the air. This not only improves users' mental performance, but in a medical segment may help those suffering with respiratory disorders that are sensitive to changes in air quality.

In embodiments, a modular design structure allows for various design elements to be mounted to the core photobioreactor, changing the design and outward appearance while maintaining air purification and HVAC integrations as described by the system design elements.

According to an embodiment, a biofiltration device may include: an air inlet that receives untreated air; a bioreactor comprising a bio-organism; an air bubble mixing system that receives the untreated air and diffuses the untreated air into the bioreactor, wherein the bio-organism is configured to consume CO₂ via photosynthesis and to release treated air; and an air outlet that receives the treated air from the bioreactor and expels the treated air.

In one embodiment, the biofiltration device may also include a pressurization pump that pressurizes the untreated air before it is received by the air bubble mixing system.

In one embodiment, the biofiltration device may also include a temperature sensor that measures a temperature of the bio-organism; a pH sensor that measures a pH of the bio-organism; a light source that provides light to the bio-organism; a heater that controls a temperature of the bio-organism in the bioreactor; and a controller that receives the temperature of the bio-organism, the pH of the bio-organism, and controls operation of the light source and the heater based on the temperature and pH.

In one embodiment, the bio-organism may include algae.

In one embodiment, the biofiltration device may also include a filtration device, wherein the treated air passes through the filtration device before it is expelled through the air outlet.

In one embodiment, the bioreactor may include a plurality of photoreactive tubes connected in series, wherein each photoreactive tube may include a U-shaped bend.

In one embodiment, the biofiltration device may also include an inlet air sensor that measures an inlet air quality of the untreated air; an outlet air sensor that measures an outlet air quality of the treated air; and a controller that controls an air injection system based on a difference between the outlet air quality and the inlet air quality.

In one embodiment, the untreated air may include conditioned air from an enclosed area, and the treated air may be expelled air to the enclosed area.

According to another embodiment, a system may include: a heating, ventilation, and air conditioning (HVAC) system comprising an air valve that controls a flow of untreated air, wherein the air is conditioned air from an enclosed area; and a biofiltration device comprising: a controller that interfaces with the air valve and controls the air valve to open or close; an air inlet that receives the untreated air from the air valve when the air valve is in an open position; a bioreactor comprising a bio-organism; an air bubble mixing system that receives the untreated air and diffuses the untreated air into the bioreactor, wherein the bio-organism is configured to consume CO₂ via photosynthesis and to release treated air; and an air outlet that receives the treated air from the bioreactor and expels the treated air to the HVAC system. For example, the treated air may be returned to the enclosed area.

In one embodiment, the controller controls a duty cycle of the air valve to be open or closed based on an air quality of the untreated air.

In one embodiment, the controller controls a duty cycle of the air valve to be open or closed based on an efficiency of the biofiltration device.

In one embodiment, the biofiltration device may further include a pressurization pump that pressurizes the untreated air before it is received by the air bubble mixing system.

In one embodiment, the biofiltration device may also include: a temperature sensor that measures a temperature of the bio-organism; a pH sensor that measures a pH of the bio-organism; a light source that provides light to the bio-organism; and a heater that controls a temperature of the bio-organism in the bioreactor. The controller receives the temperature of the bio-organism, the pH of the bio-organism, and controls operation of the light source and the heater based on the temperature and pH.

In one embodiment, the bio-organism may include algae.

In one embodiment, the bioreactor may further include a plurality of photoreactive tubes connected in series, wherein each photoreactive tube may include a U-shaped bend.

In one embodiment, the biofiltration device may be integrated into a wall or façade of a structure.

According to another embodiment, a method may include: (1) receiving, at an air inlet of a biofiltration device, untreated air; (2) pressurizing, by a pressurization pump, the untreated air; (3) diffusing, by an air bubble system, the pressurized untreated air into a bio-organism in a bioreactor, wherein the bio-organism consumes CO₂ in the untreated air via photosynthesis and releases treated air; and (4) expelling, via an air outlet and from the bioreactor, the treated air.

In one embodiment, the method may also include: receiving, by a controller, a temperature of the bio-organism and a pH of the bio-organism; and controlling, by the controller, a light source to provides light to the bio-organism and a heater to heat the bio-organism in the bioreactor based on the temperature and the pH.

In one embodiment, the method may also include: receiving, by a controller, an inlet air quality of the untreated air; receiving, by the controller, an outlet air quality of the treated air; and controlling, by the controller, an air injection system based on a difference between the outlet air quality and the inlet air quality.

In one embodiment, the method may also include: receiving, by a controller, an inlet air quality of the untreated air at an air valve of a heating, ventilation, and air conditioning (HVAC) system, wherein the untreated air comprises conditioned air from an enclosed area; receiving, by the controller, an outlet air quality of the treated air; and controlling, by the controller, a duty cycle of the air valve of a heating, ventilation, and air conditioning (HVAC) system based on the difference in air quality between the outlet air quality and the inlet air quality, and the treated air is expelled to the enclosed area.

### BRIEF DESCRIPTION OF THE DRAWINGS

For a more complete understanding of the present invention, the objects and advantages thereof, reference is now made to the following descriptions taken in connection with the accompanying drawings in which:

FIG. 1 depicts a block diagram of a system for cyanobacteria indoor air biofiltration to reduce air handling energy consumption according to an embodiment.

FIG. 2 depicts a method for cyanobacteria indoor air biofiltration to reduce air handling energy consumption according to an embodiment.

FIG. 3 depicts a method for integrating a cyanobacteria indoor air biofiltration device with a HVAC system according to an embodiment.

### DETAILED DESCRIPTION OF PREFERRED EMBODIMENTS

Systems and methods for cyanobacteria indoor air biofiltration to reduce air handling energy consumption are disclosed.

The following terms and phrases are used throughout this disclosure. Photobioreactor systems are water-holding system that incorporates some type of light source to provide photonic energy to a photosynthetic organism. These organisms, such as algae or cyanobacteria, use light to produce energy through photosynthesis. Photobioreactors are used for various applications, including the production of biofuels, pharmaceuticals, cosmetics, and food products.

Electro-flocculation (or electrocoagulation) is a water treatment process that uses electric current to remove algae from water. It is an electrochemical method that involves the generation of coagulants in situ by dissolving sacrificial anodes made of metals.

A swirl pot, swirl filter, or radial flow settler is a type of mechanical filtration system used to remove algae from the water. The swirling motion causes algae to move towards the outer edges of the pot due to centrifugal force. As the water spirals downwards, the heavier algae lose their kinetic energy and settle at the bottom of the pot.

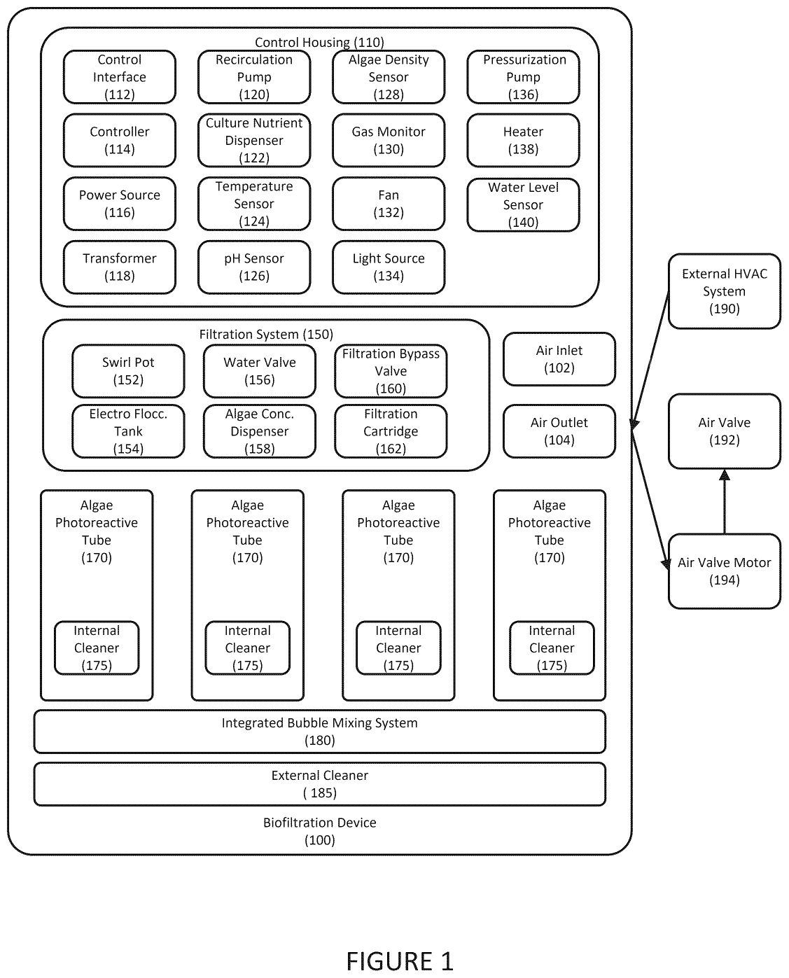

FIG. 1 depicts a block diagram of a system for cyanobacteria indoor air biofiltration to reduce air handling energy consumption according to an embodiment. Biofiltration device 100 may include control housing 110, filtration system 150, algae photoreactive tubes 170, integrated air bubble mixing system 180, and external cleaner 185.

Control housing 110 may be a part of biofiltration device 100 that includes components that are generally directed to the operation of biofiltration device 110. For example, control housing 110 may include control interface 112, controller 114, power source 116, transformer 118, recirculation pump 120, culture nutrient dispenser 122, temperature sensor 124, pH sensor 126, algae density sensor 128, input and output gas monitors 130, fan 132, light source 134, air pressurization pump 136, heater 138 and water sensor 140.

Control interface 112 may provide a mechanism for accessing controller 114, which may control the operation of biofiltration device 100. Control interface 112 may be touch-based interface (e.g., a touchscreen), a keypad, a wireless interface (e.g., accessible by a wireless electronic device, etc.).

Control interface 112 may allow an external user or system to access controller 114. For example, control interface 112 may interface with external HVAC systems 190 using a wireless, wired, or integrated interface that communicates with air valve motor 194 that may control air valve 192. For example, controller 114 may receive a signal from air valve motor 164 via control interface 112 and may then calculate the photobioreactor system efficiency and the HVAC outdoor air valve requested demand. It may then pass a control signal to air valve motor 194 to control the operation of air valve 194 to open or close, to provide or increase the flow of air to biofiltration device 100, or to reduce or stop the flow of air to biofiltration device 100.

Efficiency may be achieved by reducing its required duty.

Controller 114 may be a microprocessor or application-specific integrated circuit device that may control the operation of biofiltration device 100. Controller 114 may execute one or more computer programs that may include algorithms for controlling or interfacing with, for example, user interface 112, power source 116, transformer 118, recirculation pump 120, swirl pot 152, algae concentrate dispenser 158, algae density sensor 128, input and output gas monitors 130, fan 132, light source 134, culture nutrient dispenser 122, pH sensor 126, temp sensor 124, water level sensor 140, air pressurization pump 136, heater 138, external HVAC system 190, and air valve 192.

Controller 114 may further execute one or more computer program that may receive data from water level sensor 140, temperature sensor 124, pH sensor 126, gas monitor(s) 130, algae density sensor 128, external HVAC system 190, and/or air valve 192, and may issue control signals to control heater 138, recirculation pump 120, algae concentrate dispenser 158, light source 134, air pressurization pump 136, internal cleaners 175, external cleaners 185, filtration bypass valve 160, culture nutrient dispenser 122, external HVAC system 190, and air valve 192.

In one embodiment, air may be received by biofiltration device 110 at air inlet 102 from external HVAC system 190. For example, air may be received from the HVAC return air (where the HVAC is sucking up air from an enclosed area). In one embodiment, outdoor air may also be received.

Treated air may be discharged via air outlet 104.

Power source 116 may provide power to the components of biofiltration device 100. Power source 116 may be connected to an external power source (e.g., an AC power source, a solar panel, an eternal battery, etc.), or it may be a battery.

Transformer 118 may transform the power from power source 116 into a current and voltage that may be used by the components of biofiltration device 100.

Recirculation pump 120 may circulate water/algae mix through the entire system, mixing and unifying the properties. It may pass the algae through filtration system 150 that may include, for example, swirl pot 152, electro-flocculation tank 154, water valve 156, algae concentrate dispenser 158, filtration bypass valve 160, and filtration cartridge 162.

Controller 114 may activate recirculation pump 120 to keep the system at an equilibrium in terms of temperature and algae concentrations when the logic control dictates.

Gas monitor 130 may include one or more sensors that monitor the local conditions of gases such as, but not limited to, CO₂ and other Volatile Organic Compounds (VOCs). Gas monitor 130 may use, for example, laser spectrometry to track gases and display information to the user via user interface 112. Gasses may be expelled via fan 132. The gas monitor, or a separate gas monitor (not shown) may determine the system efficiency based on the input and output gasses.

For example, gas monitor 130 may include an inlet gas monitor or sensors that monitor the local conditions of incoming dirty or untreated air, and an outlet gas monitor or sensors that monitor the local conditions of treated air.

Light source 134 may provide light for photosynthesis as well as user customizable color patterns to provide decorative lighting. Light source 134 may be any suitable light source, including light emitting diodes. Light source 134 may cover the full visible light spectrum to replicate sunlight for photosynthesis. In one embodiment, light source 134 may provide internal lighting to the algae photoreactive tubes 150 allowing for air filtration and user-selectable color choices.

Air pressurization pump 136 may be an air-based system that receives the dirty or untreated air and increases the pressure via a pump. The pressurized dirty or untreated air may then be sent to the inlet gas monitor 130 before being injected into the algae photoreactive tubes 170 via integrated air bubble mixing system 180.

For example, integrated air bubble mixing system 180 may include a cylinder of stainless steel or similar that may have a plurality of holes (e.g., micro-sized holes). As the pressurized dirty or untreated air passes through the cylinder, it exits into photoreactive tubes 170 as small bubbles. The size of the bubbles determines the amount of surface area available. In general, smaller bubbles create more surface area, and increase the air purification rate.

The size of the holes in integrated air bubble mixing system 180 may be optimized to balance air purification rate and airflow. An example hole size is about 1 micron.

Heater 138 may generate heat to keep the system at an optimal temperature for maximum algae air filtration efficiencies. The optimal temperature may depend on the organism used. An exemplary range is from about 85 degrees F. to about 105 degrees for example, for Spirulina, an optimal temperature may be around 95 degrees F. Other temperatures may be used as is necessary and/or desired.

Fan 132 may provide both electronic component cooling as well as consistent airflow for air pressurization pump 136 to take in dirty or untreated air from the environment for biofiltration.

Water level sensor 140 may determine if there is adequate water volume in the algae photoreactive tubes 170 for the system to operate. In operation it also monitors the system for water evaporation and leakage and then notifies the controller 114 and ultimately the control interface 112 via a warning.

pH sensor 126 may monitor the algae/water pH levels to help the controller 114 determine system performance and need for nutrients.

Temperature sensor 124 may monitor the algae/water temperature to help the controller 114 determine system performance and need for heater 138 activation or deactivation.

Algae density sensor 128 may monitor the ratio of algae to water to help the controller 114 determine system performance and need for filtration or service.

Culture nutrient dispenser 122 may be a container that may or may not be fitted to embodiments to automate the water/algae nutrient and pH control by dispensing nutrients into the water/algae mix as needed and calculated by controller 114 and pH sensor 126.

Filtration system 150 may be responsible for algae density control and final offput gas filtration, it may include electro-flocculation tank 154, filtration cartridge 162, filtration bypass valve 160, and water valve 156.

Electro-flocculation tank 154 may be activated to use electrical currents with cathodes and anodes to cause algae to become attracted together in larger clusters to facilitate participation and removal from biofiltration device 100.

Water valve 156 may be used to fill the system with filtered water on setup and then to refill biofiltration device 100 as required. Water valve 156 may also function as a drain when biofiltration device 100 needs to be decommissioned or serviced.

Filtration cartridge 162 may include one or more user serviceable filters that may reduce algae density concentrations as well as act as a final purification for photobioreactor treated outlet air. These filters may be at the micron level to remove the microalgae strain from the fluid. The fluid filters may have a large surface area to reduce backpressure on fluid flow and may be cylindrical in shape.

In one embodiment, outlet air filters may be incorporated into the fluid filter. Unincorporated, the outlet air filter may capture any remaining moisture in the outlet air and provide a final particulate filtration consistent with high efficiency particulate air filters (HEPA).

Filtration bypass valve 160 may control water passage through filtration cartridge 162 and may be activated as conditions dictate to further control algae bloom densities.

Swirl pot 152 may separate dense algae blooms via a mechanical process to then be stored in algae concentrate dispenser 158 for later use. In one embodiment, swirl pot 152 may use a mechanical process, such as centrifugal separation (i.e., spinning) to separate the dense algae blooms.

In one embodiment, swirl pot 152 may drain into electro-flocculation tank 154 via gravitational settling, that may then use electric currents to cause the algae to flock together and become denser. The algae may then settle further into algae concentrate dispenser 158.

Algae concentrate dispenser 158 may provide an interface by which condensed algae from algae photoreactive tubes 170 may be output.

In one embodiment, certain elements depicted in biofiltration device 100 may be located externally to biofiltration device 100. For example, swirl pot 152, controls, pumping, and algae density sensor 128 may be located externally, thereby allowing for flexibility in the photobioreactor design by not integrating the components.

In particular, multiple biofiltration devices 100 may be provided in series. This allows for scalability by simply adding in more connections and length into the system design without drastically affecting swirl pot 158, controls, pumping, and sensing system.

Biofiltration device 100 may have some or all its components located within building walls, façades, etc.

Although embodiments may refer to the size of systems (e.g., compact scale, large scale, etc.) or applications (e.g., residential, medical, commercial, etc.), it should be recognized that these terms are used to describe the different embodiments relative to each other and are not limiting. Any embodiment may be used in any size, and for any purpose as is necessary and/or desired.

Biofiltration device 100 may further include a plurality of algae photoreactive tubes 170, each of which may include internal cleaner 175, external cleaner 185, and integrated air bubble mixing system 180.

Algae photoreactive tubes 170 may be clean containment vessels that may be linked together to create a volume for water/algae mix to pass dirty or untreated air through while collecting light spectrum to use photosynthesis in air biofiltration. In one embodiment, algae photoreactive tubes 170 may have internal coatings to reduce friction and algae buildup inside the tubing. Algae photoreactive tubes 170 may be made or ultraviolet (UV) resistance materials with a high resistance to shattering or cracking. An example material is polycarbonate.

In one embodiment, algae photoreactive tubes 170 may be provided in series design with accelerative U-bends that increase the speed of the fluid by reducing its cross section, creating an anti-settling effect for the algae. These U-bends may incorporate integrated air bubble mixing system 180 in the lower bends.

Integrated air bubble mixing system 180 may receive dirty or untreated air after being pressurized by the air pressurization pump 136 to then diffuse the dirty or untreated air into the water/algae mixture within the algae photoreactive tubes 170.

In embodiments, HVAC Communication and Outdoor Air Control may have mechanical fail-safe devices built into the controller that ensure in typical failure modes, such as: power loss, algae culture malfunction, sensor failure, system negligence, or improper use, the device will revert to default HVAC Outdoor air Valve fresh air control systems. For example, controller 114 may be programmed with these modes.

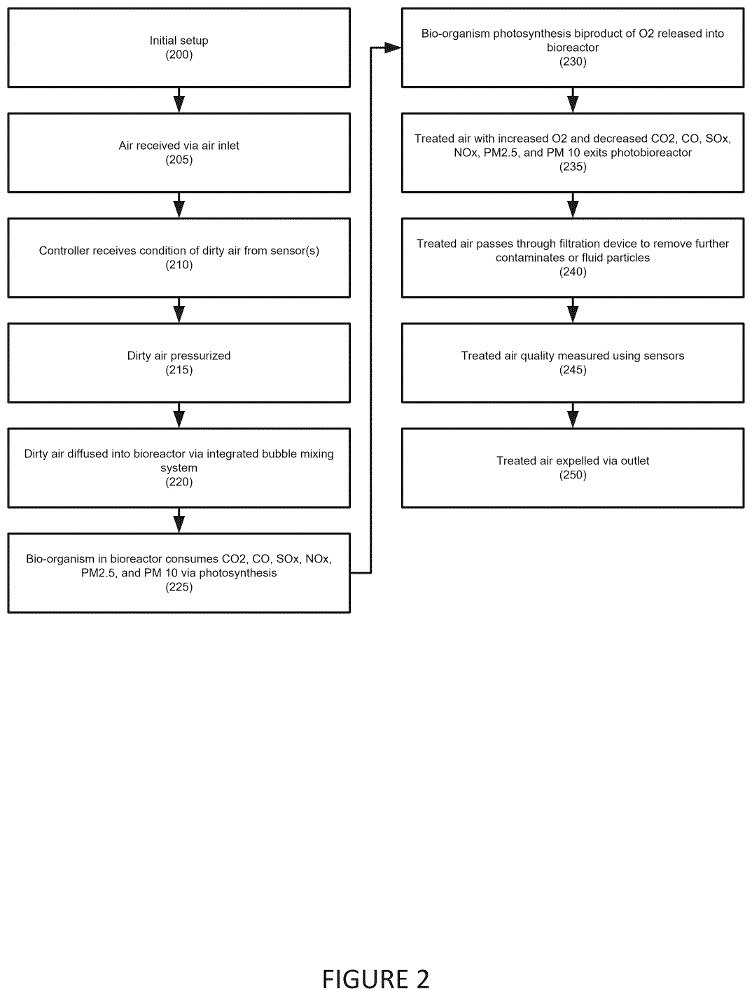

Referring to FIG. 2, a method for cyanobacteria indoor air biofiltration to reduce air handling energy consumption is disclosed according to an embodiment.

In step 200, the biofiltration device may be set up. For the first set up, the initial algae culture and nutrients may be injected into the system, such as into algae photoreactive tubes. The algae and solution may be mixed by a recirculation pump to achieve a homogeneous mixture. The algae density may be measured by an algae density sensor, and a determination of acceptability may be made by the controller.

In step 205, once the algae density is acceptable, the recirculation pump stops, the biofiltration device may receive dirty or untreated air. The dirty or untreated air may be received via air inlet and routed to inlet gas monitor. For example, the dirty or untreated air may be air that is conditioned (e.g., is at a certain temperature and humidity level) within an enclosed area, such as a room, a building, a vehicle, etc.

In step 210, a controller may receive the condition of the dirty or untreated air. For example, the controller may receive the condition of the dirty or untreated air from an inlet gas monitor, which may monitor the levels of CO₂ and other Volatile Organic Compounds (VOCs) in the dirty or untreated air. The gas monitor may use, for example, laser spectrometry to track gases and may display information on the condition of the dirty or untreated air to the user via user interface.

In step 215, the controller may pressurize the dirty or untreated air via an air pressurization system. For example, the controller may control an air pressurization pump to pressurize the dirty or untreated air. This allows the dirty or untreated air to be injected via an integrated air bubble mixing system into one or more algae photoreactive tubes.

In step 220, the pressurized dirty or untreated air may be diffused into one or more algae photoreactive tubes via an integrated air bubble mixing system. The injection pressure may be monitored and controlled by the controller to adjust for changing system requirements.

For example, in response to the dirty or untreated air quality being low, the controller may increase the injection pump speed, which increases air flow, resulting in a higher air pressure.

In step 225, inside the algae photoreactive tubes, the algae living in the solution may remove contaminants in the dirty or untreated air such as CO₂ and other VOCs using photosynthesis. Through this algae gas purification, chemical process gases are broken down with O₂ being released back into the algae photoreactive tube.

The algae density may increase with air filtration. Periodically or as necessary and/or desired, the recirculation pump may automatically cycle. This may be triggered by time, environmental conditions, demand, or the density sensor.

In one embodiment, the controller may periodically or as desired measure the algae density. If the algae density is determined to be unacceptable (i.e., too high), the flow of algae may be diverted to a filtration system until the algae density sensor reads acceptable levels.

The filtered algae may be stored in a filtration cartridge or algae concentration dispenser. When the filter is full, the filter may be replaced by user or service provider.

In step 230, a bio-organism photosynthesis biproduct of O₂ may be released into the algae solution.

In step 235, the treated air with increased O₂ and decreased CO₂ and other VOCs may bubble up to surface of the algae solution and to the upper portions of the algae photoreactive tubes.

In step 240, the treated air may exit the algae photoreactive tubes and pass through a filtration system, such as filtration cartridges, to remove further contaminants or fluid particles. The treated air may then be sent to an outlet gas monitor.

In step 245, the controller may receive the conditions of the treated air from an outlet gas monitor. The outlet gas monitor may include one or more sensors that monitor the local conditions of gases such as, but not limited to, CO₂ and other VOCs. The outlet gas monitor may use, for example, laser spectrometry to track gases and display information to the user via user interface.

The controller may display the outlet gas conditions and the change in air quality in a user interface, either locally, via an application on a remote electronic device, etc. The controller may also determine if any conditions are outside of acceptable levels, and may increase system throughput by adjusting factors such as light intensity using a light source, temperature using a heater, culture nutrient concentration using a culture nutrient dispenser, circulation using the recirculation pump, pressure using a pressurization pump, and filtration using the filtration system, etc.

In step 250, the treated air may be expelled via an outlet using a fan. The air may be expelled to the enclosed area.

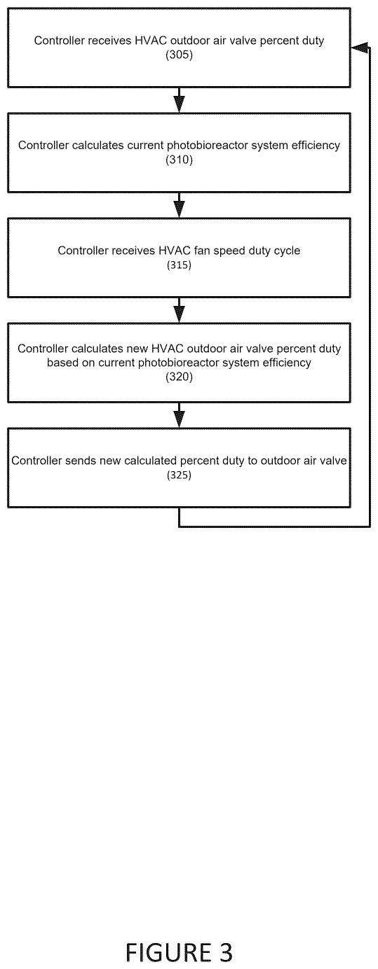

Referring to FIG. 3, a method for integrating a cyanobacteria indoor air biofiltration device with a HVAC system is provided according to an embodiment.

In step 305, a controller may receive, via a controller interface, an HVAC outdoor air valve percent duty. This may air valve on the HVAC-side that allows outside air to come into the area, such as a building.

In step 310, the controller may calculate a current biofiltration system efficiency in terms of air purification flowrate. For example, the controller may receive data from sensors, such as the inlet air quality, the outlet air quality, etc. and may use that data to calculate a system efficiency.

In step 315, the controller may receive, via the controller interface, the HVAC fan speed duty cycle. This data may be used by the controller to help compute system efficiency and throughput.

In step 320, a computer program may calculate a new HVAC outdoor air valve percent duty based on current photobioreactor system efficiency. For example, the controller may calculate a new HVAC outdoor air valve percent duty to be reduced by the current photobioreactor system efficiency in terms of flowrate.

In step 325, the controller may send the new calculated percent duty to the outdoor air valve. For example, the controller may intercept the signal between the outdoor air valve and HVAC controller and modify it before reaching the outdoor air valve, the controller may communicate the new calculated percent duty to the HVAC controller directly, etc. The new calculated percent duty may cause the motor to adjust the opening of the air valve.

During the communication process, if any errors or values outside of expectation are encountered, the controller may activate its defaults. For example, the controller may set the external HVAC system and/or the air valve to the default HVAC system requested values, and may also put the biofiltration device into a culture preservation mode. It may then notify the user via the control interface of a need for service.

Hereinafter, general aspects of implementation of the systems and methods of embodiments will be described.

Embodiments of the system or portions of the system may be in the form of a “processing machine,” such as a general-purpose computer, for example. As used herein, the term “processing machine” is to be understood to include at least one processor that uses at least one memory. The at least one memory stores a set of instructions. The instructions may be either permanently or temporarily stored in the memory or memories of the processing machine. The processor executes the instructions that are stored in the memory or memories in order to process data. The set of instructions may include various instructions that perform a particular task or tasks, such as those tasks described above. Such a set of instructions for performing a particular task may be characterized as a program, software program, or simply software.

In one embodiment, the processing machine may be a specialized processor.

In one embodiment, the processing machine may be a cloud-based processing machine, a physical processing machine, or combinations thereof.

As noted above, the processing machine executes the instructions that are stored in the memory or memories to process data. This processing of data may be in response to commands by a user or users of the processing machine, in response to previous processing, in response to a request by another processing machine and/or any other input, for example.

As noted above, the processing machine used to implement embodiments may be a general-purpose computer. However, the processing machine described above may also utilize any of a wide variety of other technologies including a special purpose computer, a computer system including, for example, a microcomputer, mini-computer or mainframe, a programmed microprocessor, a micro-controller, a peripheral integrated circuit element, a CSIC (Customer Specific Integrated Circuit) or ASIC (Application Specific Integrated Circuit) or other integrated circuit, a logic circuit, a digital signal processor, a programmable logic device such as a FPGA (Field-Programmable Gate Array), PLD (Programmable Logic Device), PLA (Programmable Logic Array), or PAL (Programmable Array Logic), or any other device or arrangement of devices that is capable of implementing the steps of the processes disclosed herein.

The processing machine used to implement embodiments may utilize a suitable operating system.

It is appreciated that in order to practice the method of the embodiments as described above, it is not necessary that the processors and/or the memories of the processing machine be physically located in the same geographical place. That is, each of the processors and the memories used by the processing machine may be located in geographically distinct locations and connected so as to communicate in any suitable manner. Additionally, it is appreciated that each of the processor and/or the memory may be composed of different physical pieces of equipment. Accordingly, it is not necessary that the processor be one single piece of equipment in one location and that the memory be another single piece of equipment in another location. That is, it is contemplated that the processor may be two pieces of equipment in two different physical locations. The two distinct pieces of equipment may be connected in any suitable manner. Additionally, the memory may include two or more portions of memory in two or more physical locations.

To explain further, processing, as described above, is performed by various components and various memories. However, it is appreciated that the processing performed by two distinct components as described above, in accordance with a further embodiment, may be performed by a single component. Further, the processing performed by one distinct component as described above may be performed by two distinct components.

In a similar manner, the memory storage performed by two distinct memory portions as described above, in accordance with a further embodiment, may be performed by a single memory portion. Further, the memory storage performed by one distinct memory portion as described above may be performed by two memory portions.

Further, various technologies may be used to provide communication between the various processors and/or memories, as well as to allow the processors and/or the memories to communicate with any other entity, i.e., so as to obtain further instructions or to access and use remote memory stores, for example. Such technologies used to provide such communication might include a network, the Internet, Intranet, Extranet, a LAN, an Ethernet, wireless communication via cell tower or satellite, or any client server system that provides communication, for example. Such communications technologies may use any suitable protocol such as TCP/IP, UDP, or OSI, for example.

As described above, a set of instructions may be used in the processing of embodiments. The set of instructions may be in the form of a program or software. The software may be in the form of system software or application software, for example. The software might also be in the form of a collection of separate programs, a program module within a larger program, or a portion of a program module, for example. The software used might also include modular programming in the form of object-oriented programming. The software tells the processing machine what to do with the data being processed.

Further, it is appreciated that the instructions or set of instructions used in the implementation and operation of embodiments may be in a suitable form such that the processing machine may read the instructions. For example, the instructions that form a program may be in the form of a suitable programming language, which is converted to machine language or object code to allow the processor or processors to read the instructions. That is, written lines of programming code or source code, in a particular programming language, are converted to machine language using a compiler, assembler or interpreter. The machine language is binary coded machine instructions that are specific to a particular type of processing machine, i.e., to a particular type of computer, for example. The computer understands the machine language.

Any suitable programming language may be used in accordance with the various embodiments. Also, the instructions and/or data used in the practice of embodiments may utilize any compression or encryption technique or algorithm, as may be desired. An encryption module might be used to encrypt data. Further, files or other data may be decrypted using a suitable decryption module, for example.

As described above, the embodiments may illustratively be embodied in the form of a processing machine, including a computer or computer system, for example, that includes at least one memory. It is to be appreciated that the set of instructions, i.e., the software for example, that enables the computer operating system to perform the operations described above may be contained on any of a wide variety of media or medium, as desired. Further, the data that is processed by the set of instructions might also be contained on any of a wide variety of media or medium. That is, the particular medium, i.e., the memory in the processing machine, utilized to hold the set of instructions and/or the data used in embodiments may take on any of a variety of physical forms or transmissions, for example. Illustratively, the medium may be in the form of a compact disc, a DVD, an integrated circuit, a hard disk, a floppy disk, an optical disc, a magnetic tape, a RAM, a ROM, a PROM, an EPROM, a wire, a cable, a fiber, a communications channel, a satellite transmission, a memory card, a SIM card, or other remote transmission, as well as any other medium or source of data that may be read by the processors.

Further, the memory or memories used in the processing machine that implements embodiments may be in any of a wide variety of forms to allow the memory to hold instructions, data, or other information, as is desired. Thus, the memory might be in the form of a database to hold data. The database might use any desired arrangement of files such as a flat file arrangement or a relational database arrangement, for example.

In the systems and methods, a variety of “user interfaces” may be utilized to allow a user to interface with the processing machine or machines that are used to implement embodiments. As used herein, a user interface includes any hardware, software, or combination of hardware and software used by the processing machine that allows a user to interact with the processing machine. A user interface may be in the form of a dialogue screen for example. A user interface may also include any of a mouse, touch screen, keyboard, keypad, voice reader, voice recognizer, dialogue screen, menu box, list, checkbox, toggle switch, a pushbutton or any other device that allows a user to receive information regarding the operation of the processing machine as it processes a set of instructions and/or provides the processing machine with information. Accordingly, the user interface is any device that provides communication between a user and a processing machine. The information provided by the user to the processing machine through the user interface may be in the form of a command, a selection of data, or some other input, for example.

As discussed above, a user interface is utilized by the processing machine that performs a set of instructions such that the processing machine processes data for a user. The user interface is typically used by the processing machine for interacting with a user either to convey information or receive information from the user. However, it should be appreciated that in accordance with some embodiments of the system and method, it is not necessary that a human user actually interact with a user interface used by the processing machine. Rather, it is also contemplated that the user interface might interact, i.e., convey and receive information, with another processing machine, rather than a human user. Accordingly, the other processing machine might be characterized as a user. Further, it is contemplated that a user interface utilized in the system and method may interact partially with another processing machine or processing machines, while also interacting partially with a human user.

It will be readily understood by those persons skilled in the art that embodiments are susceptible to broad utility and application. Many embodiments and adaptations of the present invention other than those herein described, as well as many variations, modifications and equivalent arrangements, will be apparent from or reasonably suggested by the foregoing description thereof, without departing from the substance or scope. Accordingly, while the embodiments of the present invention have been described here in detail in relation to its exemplary embodiments, it is to be understood that this disclosure is only illustrative and exemplary of the present invention and is made to provide an enabling disclosure of the invention. Accordingly, the foregoing disclosure is not intended to be construed or to limit the present invention or otherwise to exclude any other such embodiments, adaptations, variations, modifications or equivalent arrangements.

What is claimed is:

1. A biofiltration device, comprising:
an air inlet that receives untreated air;
a bioreactor comprising a bio-organism;
an air bubble mixing system that receives the untreated air and diffuses the untreated air into the bioreactor, wherein the bio-organism is configured to consume CO₂ via photosynthesis and to release treated air; and
an air outlet that receives the treated air from the bioreactor and expels the treated air.

2. The biofiltration device of claim 1, further comprising:
a pressurization pump that pressurizes the untreated air before it is received by the air bubble mixing system.

3. The biofiltration device of claim 1, further comprising:
a temperature sensor that measures a temperature of the bio-organism;
a pH sensor that measures a pH of the bio-organism;
a light source that provides light to the bio-organism;
a heater that controls a temperature of the bio-organism in the bioreactor; and
a controller that receives the temperature of the bio-organism, the pH of the bio-organism, and controls operation of the light source and the heater based on the temperature and pH.

4. The biofiltration device of claim 1, wherein the bio-organism comprises algae.

5. The biofiltration device of claim 1, further comprising a filtration device, wherein the treated air passes through the filtration device before it is expelled through the air outlet.

6. The biofiltration device of claim 1, wherein the bioreactor comprises a plurality of photoreactive tubes connected in series, wherein each photoreactive tube comprises a U-shaped bend.

7. The biofiltration device of claim 1, further comprising:
an inlet air sensor that measures an inlet air quality of the untreated air;
an outlet air sensor that measures an outlet air quality of the treated air; and
a controller that controls an air injection system based on a difference between the outlet air quality and the inlet air quality.

8. The biofiltration device of claim 1, wherein the untreated air comprises conditioned air from an enclosed area, and the treated air is returned to the enclosed area.

9. A system, comprising:
a heating, ventilation, and air conditioning (HVAC) system comprising an air valve that controls a flow of untreated air, wherein the untreated air is conditioned air from an enclosed area; and
a biofiltration device comprising:
a controller that interfaces with the air valve and controls the air valve to open or close;
an air inlet that receives the untreated air from the air valve when the air valve is in an open position;
a bioreactor comprising a bio-organism;
an air bubble mixing system that receives the untreated air and diffuses the untreated air into the bioreactor, wherein the bio-organism is configured to consume CO₂ via photosynthesis and to release treated air; and
an air outlet that receives the treated air from the bioreactor and expels the treated air to the enclosed area.

10. The system of claim 9, wherein the controller controls a duty cycle of the air valve to be open or closed based on an air quality of the untreated air.

11. The system of claim 9, wherein the controller controls a duty cycle of the air valve to be open or closed based on an efficiency of the biofiltration device.

12. The system of claim 9, wherein the biofiltration device further comprises a pressurization pump that pressurizes the untreated air before it is received by the air bubble mixing system.

13. The system of claim 9, wherein the biofiltration device further comprises:
a temperature sensor that measures a temperature of the bio-organism;
a pH sensor that measures a pH of the bio-organism;
a light source that provides light to the bio-organism; and
a heater that controls a temperature of the bio-organism in the bioreactor;
wherein the controller receives the temperature of the bio-organism, the pH of the bio-organism, and controls operation of the light source and the heater based on the temperature and pH.

14. The system of claim 9, wherein the bio-organism comprises algae.

15. The system of claim 9, wherein the bioreactor further comprises a plurality of photoreactive tubes connected in series, wherein each photoreactive tube comprises a U-shaped bend.

16. The system of claim 9, wherein the biofiltration device is integrated into a wall or façade of a structure.

17. A method, comprising:
receiving, at an air inlet of a biofiltration device, untreated air;
pressurizing, by a pressurization pump, the untreated air;
diffusing, by an air bubble system, the pressurized untreated air into a bio-organism in a bioreactor, wherein the bio-organism consumes CO₂ in the untreated air via photosynthesis and releases treated air; and
expelling, via an air outlet and from the bioreactor, the treated air.

18. The method of claim 17, further comprising:
receiving, by a controller, a temperature of the bio-organism and a pH of the bio-organism; and
controlling, by the controller, a light source to provide light to the bio-organism and a heater to heat the bio-organism in the bioreactor based on the temperature and the pH.

19. The method of claim 17, further comprising:
receiving, by a controller, an inlet air quality of the untreated air;
receiving, by the controller, an outlet air quality of the treated air; and
controlling, by the controller, an air injection system based on a difference between the outlet air quality and the inlet air quality.

20. The method of claim 19, further comprising:
receiving, by a controller, an inlet air quality of the untreated air at an air valve of a heating, ventilation, and air conditioning (HVAC) system, wherein the untreated air comprises conditioned air from an enclosed area;
receiving, by the controller, an outlet air quality of the treated air; and
controlling, by the controller, a duty cycle of the air valve of a heating, ventilation, and air conditioning (HVAC) system based on the difference in air quality between the outlet air quality and the inlet air quality;
wherein the treated air is expelled to the enclosed area.
